SEQUENCE LISTING (9 sequences)

SEQ ID NO: 1 - DNA, 31 bp, Artificial
  Primer
gccgatatcatggtgagcaagggcgaggagc

SEQ ID NO: 2 - DNA, 39 bp, Artificial
  Primer
cgcgatatccttgtacagctcgtccatgccgagagtgat

SEQ ID NO: 3 - DNA, 30 bp, Artificial
  Primer
cagcgctgatggtgagcaagggcgaggagc

SEQ ID NO: 4 - DNA, 31 bp, Artificial
  Primer
cagcgcttgtacagctcgtccatgccgagag

SEQ ID NO: 5 - DNA, 37 bp, Artificial
  Primer
ggccgcccgggccccaccggtcgccaccatggtgagc

SEQ ID NO: 6 - DNA, 40 bp, Artificial
  Primer
ggcccggggggccttgtacagctcgtccatgccgagagtg

SEQ ID NO: 7 - DNA, 33 bp, Artificial
  Primer
gtcactcgagatggtggctttcaaaggggtctg

SEQ ID NO: 8 - DNA, 36 bp, Artificial
  Primer
gcatcccgggtcatactgaagacaatacctctccag

SEQ ID NO: 9 - DNA, 33 bp, Artificial
  Primer
gtcactcgagatgagtgacagacccacagcaag

(19) United States
(12) Patent Application Publication
(10) Pub. No.: US 2009/0029388 A1
(43) Pub. Date: Jan. 29, 2009

(54) Detection of Antibodies

(76) Inventor: David Beeson, Oxford (GB)

Correspondence Address: ROTHWELL, FIGG, ERNST & MANBECK, P.C., 1425 K STREET, N.W., SUITE 800, WASHINGTON, DC, 20005, US

(21) Appl. No.: 12/097,399

(22) PCT Filed: Dec. 14, 2006
(86) PCT No.: PCT/GB2006/050455

§ 371 Date: Sep. 19, 2008

(30) Foreign Application Priority Data
Dec. 15, 2005 (GB) 0525541.9

(51) Int. Cl. G01N 33/53

(52) U.S. Cl. 435/7.1

(57) ABSTRACT

The present invention relates to a method for detecting antibodies against a target antigen in a sample which comprises contacting the sample with labelled target antigen, subjecting the sample to immunoprecipitation to precipitate antibodies in the sample and detecting the presence of antibodies against the target antigen in the sample by means of the presence of labelled target antigen in the immunoprecipitate, wherein the labelled target antigen is a fusion protein comprising the target antigen and a fluorescent protein label and the presence of labelled target antigen in the immunoprecipitate is detected by means of the fluorescence of the fluorescent label. The method is particularly suitable for use where the target antigen is an autoantigen and can also be used to identify autoantigens implicated in a particular autoimmune disorder by screening serum samples from patients with a clinical phenotype indicative or suggestive of an autoimmune disorder and suitable controls. The target protein may be from the cys-loop acetyl choline receptor ion channel gene superfamily, the voltage-gated calcium, sodium or potassium ion channel gene superfamily, the glutamate receptor gene family, a receptor tyrosine kinase, or other membrane associated channels such as aquaporin gene family.

### FIELD OF THE INVENTION

This invention relates to the detection of antibodies and in particular to a method which is particularly suitable for detecting and/or identifying autoantibodies.

### BACKGROUND TO THE INVENTION

Radioimmunoprecipitation of a target antigen is a standard method for the detection of autoantibodies in a sample such as serum and in this detection method the target antigen must be directly or indirectly radio-labelled. For example, the target antigen may be directly labelled by iodination with ¹²⁵I or by incorporation of ³⁵S-methionine, or the target antigen may be indirectly labelled through binding to a radio-labelled high-affinity ligand, such as ¹²⁵I-α-bungarotoxin or ¹²⁵I-α-dendrotoxin. Working with radioactivity involves inherent dangers and these methods require precautions to be taken against the dangers of using radioactivity.

In many cases autoantibody specificity requires that the antigen be in its native conformation and in this case labelling with antigen-specific toxins is often used. However, the use of high affinity radio-labelled ligands may mask some autoantibody binding sites on the target antigen.

Similar problems may apply to the use of radioimmunoprecipitation of antibodies generally in samples such as serum, particularly in cases where antibody specificity requires the antigen to be in its native conformation.

It is also known to carry out immunoprecipitation assays using a wide variety of labels other than radio-labels. For example, the target antigen may be labelled by incubation with a fluorescer, such as fluorescein isothiocyanate (FITC) and rhodamine compounds, to couple the antigen to the fluorescer. The target antigen may alternatively be tagged with other labels, including enzymes such as alkaline phosphatise and horseradish peroxidise; chemiluminescers such as isoluminol; and the like. As with the use of high affinity radio-labelled ligands, the use of these other labels may mask some autoantibody binding sites on the target antigen.

Accordingly, there is a need for an improved method for the detection of antibodies in a sample such as serum which does not require the use of a radio-labelled antigen or a radio-labelled ligand. There is a particular need for such a method which is highly sensitive and specific and which is suitable for the detection of autoantibodies in a sample such as serum.

Lennon et al. J Exp Med (2005) 202 (4): 473-7 and Weinshenker et al. Dis Markers. (2006) 22 (4):197-206 describe immunohistochemical techniques to detect the presence of autoantibodies to the water channel aquaporin-4 (AQP4). They make use of aquaporin 4 labelled with an EGFP-tag for co-localisation of immunostaining on cells, or for analysis of immunoprecipitation on western blots. These are standard techniques for analysis of antibody binding and EGFP fluorescence is not used as a read out for detection of the autoantibodies.

### SUMMARY OF THE INVENTION

According to one aspect, the present invention provides a method for detecting antibodies against a target antigen in a sample which comprises contacting the sample with labelled target antigen, subjecting the sample to immunoprecipitation to precipitate antibodies in the sample and detecting the presence of antibodies against the target antigen in the sample by means of the presence of labelled target antigen in the immunoprecipitate, wherein the labelled target antigen is a fusion protein comprising the target antigen and a fluorescent protein label and the presence of labelled target antigen in the immunoprecipitate is detected by means of the fluorescence of the fluorescent protein label.

The use of a fusion protein whereby the fluorescent protein label is incorporated directly in the biological synthesis (i.e. translation) of the target antigen is advantageous because the fluorescent tag can be incorporated at chosen positions within the antigen and because the labelled antigen can maintain immunogenic conformation. Further, when assaying no external label and therefore no purification is required. In particular a radioactive label is not required. The use of such an intrinsically fluorescence-labelled antigen is proving to be a highly efficient method.

The method according to the invention can be qualitative, i.e. it can simply be used to detect the presence or absence of antibodies against the target antigen in a sample. However, the method is preferably quantitative in that the amount of antibody is determined by quantitating the fluorescence in the immunoprecipitate. The method can be applied to detecting antibodies against any target antigen in a sample, preferably a serum sample, but the method is particularly suitable for use where the target antigen is an autoantigen.

The method can also be used to identify autoantigens implicated in a particular autoimmune disorder. Thus, according to another aspect, the present invention provides a method for identifying autoantigens implicated in an autoimmune disorder which comprises screening serum samples from patients with a clinical phenotype indicative or suggestive of an autoimmune disorder and suitable controls with a labelled putative autoantigen, subjecting the samples to immunoprecipitation to precipitate antibodies therein and identifying actual autoantigens by the presence of the labelled putative autoantigen in the immunoprecipitate, wherein the labelled putative autoantigen is a fusion protein comprising the putative autoantigen and a fluorescent protein label and the presence of labelled putative autoantigen in the immunoprecipitate is detected by means of the fluorescence of the fluorescent protein label.

According to another aspect, the invention relates to a reagent suitable for use in the above methods comprising a fusion protein comprising the target antigen and a fluorescent protein label. The target antigen may be:

a protein which is a member of the cys-loop acetyl choline receptor ion channel gene superfamily;
a protein which is a member of the voltage-gated calcium, sodium or potassium ion channel gene superfamily;
a protein which is a member of the glutamate receptor gene family; or
a receptor tyrosine kinase; or a protein which is a member of other membrane-associated channel gene families.

### DETAILED DESCRIPTION OF THE INVENTION

The present invention relates to a method which makes use of a target antigen labelled or tagged in a manner which enables it to be detected by means of fluorescence. The label is a fluorescent marker (also referred to herein as a tag) which is used to label the target antigen directly, i.e. the target protein and the fluorescent marker are formed as a fusion protein.

The target antigen is a protein which generates an immune response in an animal, the immune response involving the production of antibodies specific to the target protein.

The fluorescent marker is a protein which fluoresces under appropriate conditions, in particular when exposed to light of the appropriate wavelength. The target antigen is labelled with the fluorescent marker by being engineered so that the fluorescent tag is incorporated into or fused with the target antigen so that sufficient of the native conformational structure of the target antigen is retained that the labelled target antigen binds to antibodies raised against the native antigen.

Any molecular fluorophore can be used as the fluorescent tag provided that it can be fused with the target antigen without disrupting the antigenic properties of the target antigen. Preferred fluorescent tag proteins include those derived from the jelly fish protein known as green fluorescent protein (GFP). Further information on GFP and other fluorophores is given in the following publications:
Tsien R Y, “The Green Fluorescent Protein” Annual Reviews of Biochemistry 1998; 67:509-544
Verkhusha, V and Lukyanov, K. “The Molecular Properties and Applications of Anthoza Fluorescent Proteins and Chromophores” Nature Biotechnology 2004; 22:289-296.

Plasmid vectors encoding a wide range of fluorescent tag proteins are commercially available from various suppliers including an array of “Living Colours™ Fluorescent Proteins” available commercially from Clontech Laboratories, Inc. Similar vectors can also be obtained from other suppliers including Invitrogen and Amersham Biosciences.

Preferred fluorescent proteins derived from GFP are the red-shifted variant EGFP, the cyan shifted variant ECFP and the yellow shifted variant EYFP. Alternative fluorescent marker proteins are commercially available. EGFP is preferred as the fluorescent marker because it gives bright fluorescence combined with minimal effect on the antigenic properties of the target antigen.

The method of the present invention is applicable to the detection of antibodies against any antigen in a sample, however, the method is particularly advantageous when applied to the detection of autoantigens. For example the method can be applied to the following antigens:
proteins which are members of the cys-loop acetylcholine receptor ion channel gene superfamily, such as neuronal nicotinic AChRs, GABAA receptors, glycine receptors and the 5HT₃ receptor
proteins which are members of the voltage-gated calcium, sodium or potassium ion channel gene superfamily such as Kv1.1-Kv1.7 (or KCNA1-KCNA7)
proteins which are members of the glutamate receptor gene family such as GluR1-GluR4, kainate receptors and NMDA receptors
receptor tyrosine kinases such as muscle specific kinase (MuSK), and growth factor receptors such as fibroblast growth factor receptors, e.g. FGFR1-FGFR3.
other membrane associated channels such as aquaporin gene family

The nucleotide and/or amino acid sequences for many such antigens are available in the literature including the following:
Valenzuela et al, “Receptor tyrosine kinase specific for the skeletal muscle lineage: expression in embryonic muscle, at the neuromuscular junction, and after injury” Neuron, 1995; 15: 573-84
Beeson et al, “Primary structure of the human muscle acetylcholine receptor cDNA cloning of the γ and ε subunits” European Journal of Biochemistry, 1993; 215: 229-238
Noda et al, “Cloning and sequence analysis of calf cDNA and human genomic DNA encoding α-subunit precursor of muscle acetylcholine receptor” Nature, 1983; 305: 818-823
Luther et al, “A muscle acetylcholine receptor is expressed in the human cerebellar medulloblastoma cell line TE671” Journal of Neuroscience, 1989; 9 (3): 1082-96.
Yang et al, cDNA cloning, gene organization, and chromosomal localization of a human mercurial insensitive water channel. Evidence for distinct transcriptional units. J Biol Chem 1995; 270:22907-22913.

Although reference is made herein to a target antigen, it should be understood that it may not always be necessary to use the complete antigen and in some cases the antigen will still be recognised even if part of the structure has been lost. For example, in the case of the MuSK antigen, antigenic properties will generally be dependent on the extracellular domain of the antigen since this is the part of the antigen that is accessible for antibody binding. Conversely, for some disease markers, such as in paraneoplastic disorders and anti-Hu and anti-Yo antibodies, the antigen is located within the cell and antibodies will still bind to the wholly or partially denatured antigen. However, for complex multi-subunit proteins such as AChR, it is likely that the substantially complete antigen will be needed for antigenic properties to be retained.

The labelled target antigen will generally need to be made by appropriate engineering of the target antigen. Accordingly, a suitable construct will be prepared of DNA encoding the target antigen and DNA encoding the tag in frame therewith so that expression results in a fusion protein comprising the target antigen and the tag generally incorporated into or fused with the target antigen at the appropriate position. If not already available, DNA encoding the target antigen can be obtained by standard techniques of recombinant DNA technology.

Fluorescent tag proteins are normally available in the form of vectors including DNA encoding the tag protein suitable for incorporation into constructs with DNA encoding other proteins. The construct of DNA encoding the target antigen and DNA encoding the fluorescent tag will be incorporated in an expression vector for expression together with suitable control elements. The expression vector will then be incorporated into a suitable host cell line for production of the protein. The host cell line must be capable of producing the protein in the correct conformation so that antigenic properties are retained. Many suitable host cell lines for expression of the protein with the correct conformation are available including human cell lines and other mammalian cell lines. In some cases, insect cell lines may also be used or even bacterial expression systems such as E coli.

It is important that the fluorescent tag can be incorporated into the target antigen without affecting the antigenic properties of the target antigen. The way in which this can be done is specific to the antigen and may require some knowledge of the antigen structure. For example, it has been found that in the case of AChR, a fluorescent tag protein such as EGFP can be inserted into the cytoplasmic loop structure between transmembrane domains 3 and 4 without affecting antigenic properties. In the case of other proteins such as MuSK a fluorescent tag protein can be fused to the N- or C-terminus of the protein without affecting antigenic properties.

Determining where the fluorescent tag protein can be incorporated into or fused to the target antigen is essentially an empirical process to be undertaken on a case by case basis. As already noted, DNA encoding the target antigen is required to make a construct with DNA encoding the fluorescent tag protein for use in expressing the tagged target antigen. This implies that information concerning the antigen structure will also be available and this information will generally be sufficient to enable predictions to be made as to suitable starting points for insertion of the fluorescent marker protein.

Once the labelled target antigen has been produced, it will be necessary to confirm that the antigen has the correct conformation so that antigenic properties have been retained. The required conformation is likely to be protein specific and can be established by the binding of known conformation-specific ligands or known conformation-dependent antibodies.

As already noted, the method of the present invention is particularly applicable for the detection of autoantigens and although many autoantigens have been identified, it is believed that a wealth of autoantigenic targets remain to be discovered. The method of the present invention can be used to identify autoantigens by labelling candidate putative antigens with a tag, generally a tag protein, which is fluorescent, and then screening serum samples from particular patient groups to see whether these samples contain antibodies to the putative autoantigen. Thus groups of serum samples can be obtained from patients who have similar clinical phenotypes with characteristics indicative of an autoimmune disorder. This may be a known autoimmune disorder in which case then method can be used to identify autoantigens associated with that disorder. Alternatively, the patients may have characteristics suggestive of an autoimmune disorder but without the disorder yet having been conclusively identified as autoimmune. Thus the patients may respond to immunosuppressive therapy, generally have a fluctuating course of disease, there may be family associations with other autoimmune disorders, and they may share common HLA haplotypes. In this case, as well as identifying autoantigens, the method may assist in confirming disorders as autoimmune in nature.

If antibodies against the target antigen are present in a sample, the target antigen labelled with the tag bound to those antibodies will be precipitated in an immunoprecipitation. The fluorescence associated with the tag can then be used to detect protein precipitated in this way (qualitative determination) or the fluorescence read out can be used as a measure of the amount of protein precipitated (quantitative determination).

In one example of an immunoprecipitation, soluble extracts of a fluorescence-tagged antigen are incubated with patient sera for an appropriate period of time, usually overnight at 4° C. (typically 10-15 μl of serum to 300-500 μl of extract or less) to allow autoantibodies/antibodies to bind to their target protein. Protein A or Protein G Sepharose beads, preincubated with low IgG fetal calf serum (Sigma) to block non-specific binding, are then added to the extract/serum mix containing the tagged protein/antibody complexes, and mixed with gentle rotation for 1 to 2 hours at room temperature. The antibodies within the serum, including those that specifically bind the tagged protein, are bound by the protein A/G beads. The protein A/G Sepharose beads are then washed in a suitable buffer (typically 10 mM Tris-HCl pH 7.4, 100 mM NaCl/1 mM EDTA/1% Triton X-100) to remove any unbound tagged protein. Typically this is achieved by 3 rounds of gentle centrifugation (3000 r.p.m. in a benchtop microfuge for 1 minute), removal of the supernatant and resuspension in buffer. The protein-A/G beads, some with tagged protein attached, are collected and placed in a fluorescence reader, for example a Spectra Max Gemini XS plate reader from Molecular Devices Inc, placing the beads in conical bottomed black PCR plates (Thermo-fast 96 well black PCR plates) obtained from ABgene. The presence of specific autoantibodies/antibodies in the original serum sample is determined and quantitated using the fluorescence read-out. In the case of GFP this uses excitation at wavelength 472 nm and emission at 512 nm. The fluorescence excitation will depend upon the fluorophore/tag that is used but it would be possible to combine several different tagged proteins in the same time, for example AChR-EGFP with MuSK-DsRed2 can be combined. The sensitivity of the method is dependent on the detection device and can be considerably enhanced by using more sensitive detection devices.

Various modifications of the method could be incorporated depending upon the biology of the antigen and the tag. Different tags have different excitation/emission spectra which will require modification of the detection device in accordance with the tag but also allows the possibility of carrying several tests in the same tube as one assay. Other modifications include putting a second (non-fluorescent) tag on the target antigen which can be used for purification prior to adding the patient serum. For example, this can be applied to MuSK using purification via a poly-histidine tag and metal affinity chromatography and this enhances the signal to background ratio of the results.

The present invention can be understood in more detail which reference to the following experimental work which in turn makes reference to the accompanying drawings.

### BRIEF DESCRIPTION OF THE DRAWINGS

FIG. 1A shows results of incubation of sera from patients and controls with GFP-tagged AChR, the serum-AChR complex being brought down with protein A Sepharose beads;

FIG. 1B shows results from the same sera as FIG. 1A as determined by ¹²⁵I α-bungarotoxin binding;

FIGS. 2A and 2B show results of the same experiments as FIGS. 1A and 1B except that the protein A Sepharose beads were pre-incubated in 2% BSA;

FIG. 3 shows titration of sera from high and low myasthenia gravis patients and a healthy control with GFP-tagged AChR, the serum-AChR complex being brought down with protein A Sepharose beads;

FIG. 4 shows titration of the volume of the beads used to detect serum antibodies to GFP-tagged AChR;

FIG. 5A shows results of a fluorescence immunoprecipitation assay for a set of nine “neurological disease positive” sera showing one patient as AChR antibody positive, the serum-AChR complex being brought down with protein A Sepharose beads;

FIG. 5B shows results from the same sera as FIG. 5A as determined by an ¹²⁵I α-bungarotoxin binding assay;

FIGS. 6A and 6B show results from the same experiments as FIGS. 5A and 5B except that the protein A Sepharose beads were pre-incubated in 2% BSA;

FIG. 7 shows detection of anti-MuSK antibodies in human sera;

FIG. 8 shows results of a fluorescence immunoprecipitation assay for antibodies to AQP4 in the sera from three NMO (neuromyelitis optica) patients, one healthy control and two MS (multiple sclerosis) patients. The serum-AQP4 complex being brought down with protein-A Sepharose beads (▴) and with protein-G Sepharose beads ();

FIG. 9 shows results of a fluorescence immunoprecipitation assay for antibodies to AQP4 in the sera from patients with NMO, “other neurological diseases”, MS and healthy controls.

### EXAMPLES

### Example 1

### An Assay for Serum Antibodies to AChR Using EGFP Tagged AChR α-, γ- and ε-Subunits

### Methods

Construction of Plasmids that Express EGFP-Tagged Human AChR Subunits.

Oligonucleotides were designed to amplify 615 base pairs (a polypeptide tag of 205 amino acids) encoding the EGFP sequence tag, so that the tag fragment could be ligated “in frame” within the AChR subunit cDNA sequence. The site of insertion within the AChR subunit sequence was chosen to be within the intracellular cytoplasmic domain between transmembrane regions M3 and M4, but not within the MA amphipathic helix region.

Thus for the human AChR α subunit, oligonucleotides

5′-GCCGATATCATGGTGAGCAAGGGCGAGGAGC-3′
and
5′-CGCGATATCCTTGTACAGCTCGTCCATGCCGAGAGTGAT-3′

were used to amplify the EGFP sequence. The resulting fragment was cut with EcoR V and ligated into the α-subunit cDNA sequence at the EcoR V restriction site at position 1041. Following transformation, constructs were analysed and colonies isolated in which the EGFP insert was in the correct orientation and reading frame.

For the human AChR γ subunit, oligonucleotides

5′-CAGCGCTGATGGTGAGCAAGGGCGAGGAGC-3′
and
5′-CAGCGCTTGTACAGCTCGTCCATGCCGAGAG-3′

were used to amplify the EGFP sequence. The resulting fragment was cut with Eco47 III and ligated into the γ-subunit cDNA sequence at the Eco47 III restriction site at nucleotide position 1170. Analysis of the resultant constructs allowed expression plasmids to be isolated in which the EGFP insert was in the correct orientation and reading frame.

For the human AChR ε subunit oligonucleotides

5′-GGCCGCCCGGGCCCCACCGGTCGCCACCATGGTGAGC-3′
and
5′-GGCCCGGGGGGCCTTGTACAGCTCGTCCATGCCGAGAGTG-3′

were used to amplify the EGFP sequence. The resulting fragment was digested with Sfi I and the fragment ligated in frame into the ε-subunit cDNA sequence at the Sfi I site at position 1022. Transformants were analysed to confirm the EGFP tag insert was in the correct orientation and reading frame within the expression construct.

The EGFP tagged human AChR α-, γ- and ε-subunit cDNAs were each co-transfected the respective unlabelled remaining AChR subunits (i.e. AChR-α-EGFP with β-, δ- and ε-subunit cDNAs; AChR-ε-EGFP with α-, β-, δ-subunit cDNAs; AChR-γ-EGFP with α-, β-, δ-subunit cDNAs) to check for expression of human AChR containing the EGFP tag. Moreover, following transfection into HEK 293 cells, each construct was shown to generate functional AChR by patch clamp single channel recording and robust cell surface expression as measured by ¹²⁵I-α-bungarotoxin binding or fluorescence microscopy.

### Expression of EGFP-Tagged AChR

HEK 293 or TE 671 cells were transiently transfected using calcium phosphate, with the combinations of the following fluorescently labelled AChR subunits:

### α-EGFP, β, δ and ε-EGFP or α-EGFP, β, δ and γ-EGFP.

Soluble extracts of the AChR were made 2 days post-transfection using buffer containing 10 mM Tris-HCl pH 7.4, 100 mM NaCl/1 mM EDTA/1% Triton X-100 in a volume of 300 μl per well of a 6-well culture plate. Cells were extracted for 1 hr at 4° C. and in the presence of a protease inhibitor cocktail (Sigma). The extracts, containing the fluorescent fetal and adult forms (ε-EGFP and γ-EGFP) of the AChR were combined.

### Fluorescence Immunoprecipitation

Extracts of fluorescence-tagged antigen were incubated with patient sera overnight at 4° C. (typically 10-15 μl of serum to 300-500 μl of extract). Protein A Sepharose beads were preincubated with low IgG fetal calf serum (Sigma), then 50-100 μl added to the receptor-antibody complexes, and incubated with gentle rotation for 1-2 hours at room temperature. The beads were then washed in extraction buffer (see above) 3 times by gentle centrifugation (3000 r.p.m. in a benchtop microfuge for 1 minute), removal of the supernatant and resuspension in extract buffer.

Finally, the protein-A beads were placed in a fluorescent plate reader (Spectra Max Gemini XS, Molecular Devices) using conical bottom black PCR plates (Thermo-fast 96 well black PCR plates, ABgene) and the presence of anti-AChR antibodies in sera quantitated by fluoresce read-out using excitation at wavelength 472 nm and emission at 512 nm.

### Results

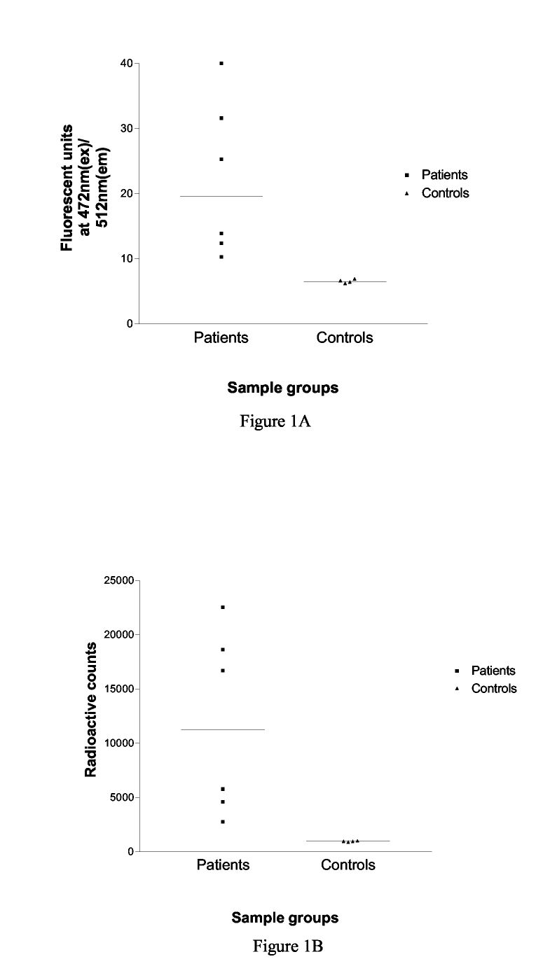

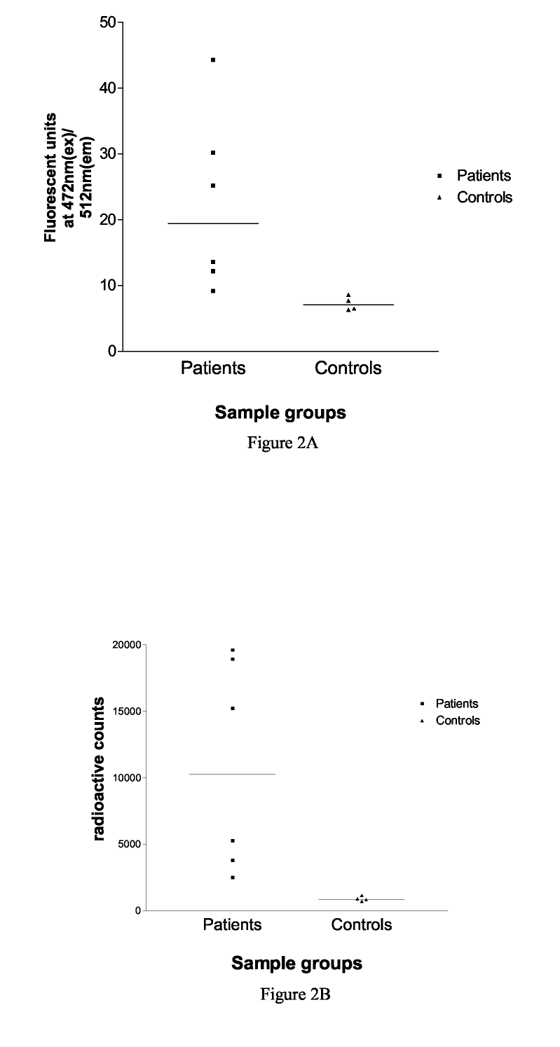

A series of AChR antibody-positive sera from patients with myasthenia gravis were used in trial assays. The sera from myasthenia gravis patients, in general, requires the antigen (AChR) to be in native conformation and thus this disorder provides a stringent test of the method. In the first series of experiments the fluorescence-tagged AChR extract was also labelled with ¹²⁵I-α-bungarotoxin to enable a direct comparison of the sensitivity of the fluorescent marker versus the standard radio-immunoprecipitation assay used for myasthenia gravis. The sera were chosen so that they covered a range of different antibody titres. FIG. 1A shows the results of the assay using a fluorescence read out and FIG. 1B the same assay with the read out in radioactive counts resulting from precipitation of ¹²⁵I-α-BuTX-bound AChR. Thus in FIG. 1, 10 μl of patient serum was incubated with 300 μl of GFP-tagged AChR extract and the serum-AChR complex brought down using 100 μl of protein A Sepharose beads. Serum antibody levels were measured by fluorescence units (A) or by γ counter (B). The patient group was chosen to consist of three high and three lower titre sera. FIG. 2 shows the same experiment as in FIG. 1 except that the beads were pre-incubated in 2% BSA prior to addition to the serum/extract mix, in an attempt to reduce background fluorescence from the beads.

FIGS. 1 and 2 show that fluorescence-tagged antigen is immunoprecipitated by sera from patients with myasthenia gravis, and that the sensitivity of the technique is comparable to radio-labelling the AChR with the specific toxin ¹²⁵I-α-bungarotoxin. In each case the read out for the fluorescence directly corresponds to the 125I-α-bungarotoxin binding precipitated.

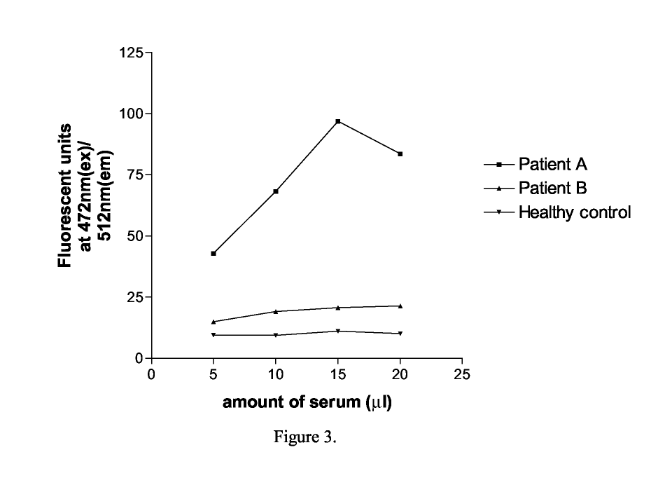

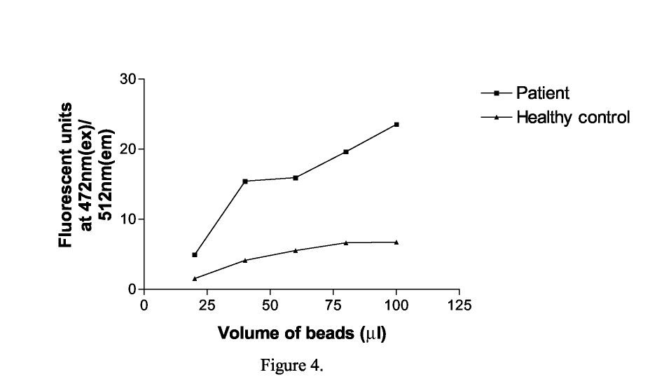

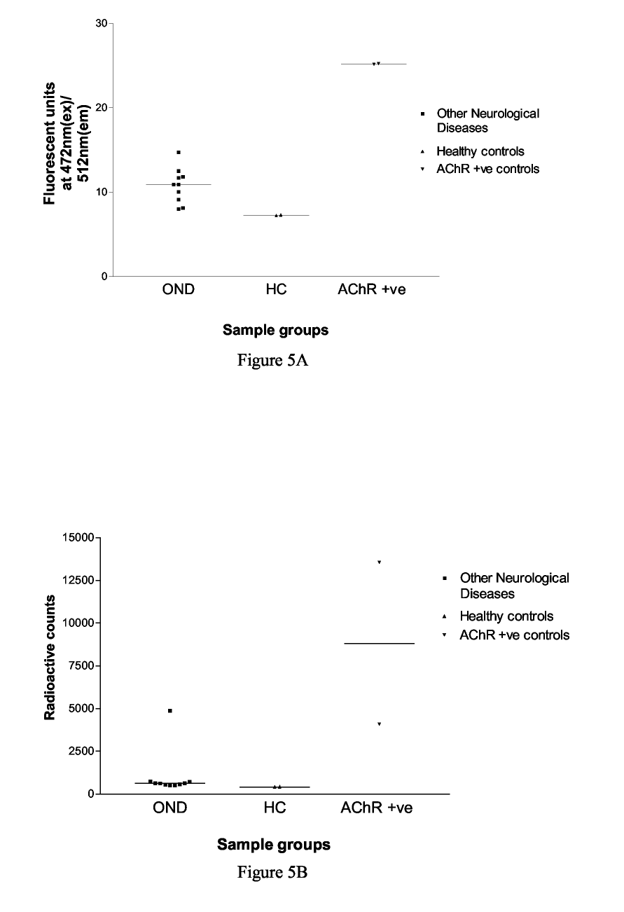

FIG. 3 shows titration of sera for a high titre MG patient (A) and a low titre MG patient (B) and a healthy control using 300 μl extract and 100 μl of protein A beads. FIG. 4 shows titration of the volume of beads used to detect serum antibodies to the AChR-EGFP. A 100 μl volume is the maximum that can be placed in 96-well plates. The fluorescence immunoprecipitation assay was also performed on a randomly selected set of sera from patients with other neurological disorders (containing one coded myasthenia gravis serum sample) (FIGS. 5A and B, and 6A and B). Although background signal from other disease controls gave an increased reading compared to healthy control samples in experiment/FIG. 5A, the assay identified the myasthenia gravis patient and a slight modification to the procedure for experiment 6A resulted in a clearer distinction between a second set of neurological disease controls and the ‘blinded’ sample from the myasthenia gravis patient. Interestingly, one AChR positive control serum gave a consistently higher relative signal for the fluorescence assay compared to the α-bungarotoxin binding assay, suggesting that in this serum sample antibodies may be present that compete for an α-bungarotoxin binding site on the AChR pentamer. For such patients, the fluorescence immunoprecipitation technique may have advantages over conventional assays that use labelled α-bungarotoxin.

FIG. 5A shows the results of a fluorescence immunoprecipitation assay in which a set of nine ‘neurological disease positive’ sera were assayed. One patient was determined as AChR antibody positive. In FIG. 5B, the same set of patients were assayed for AChR antibody positive sera using the standard ¹²⁵I-α-bungarotoxin binding assay.

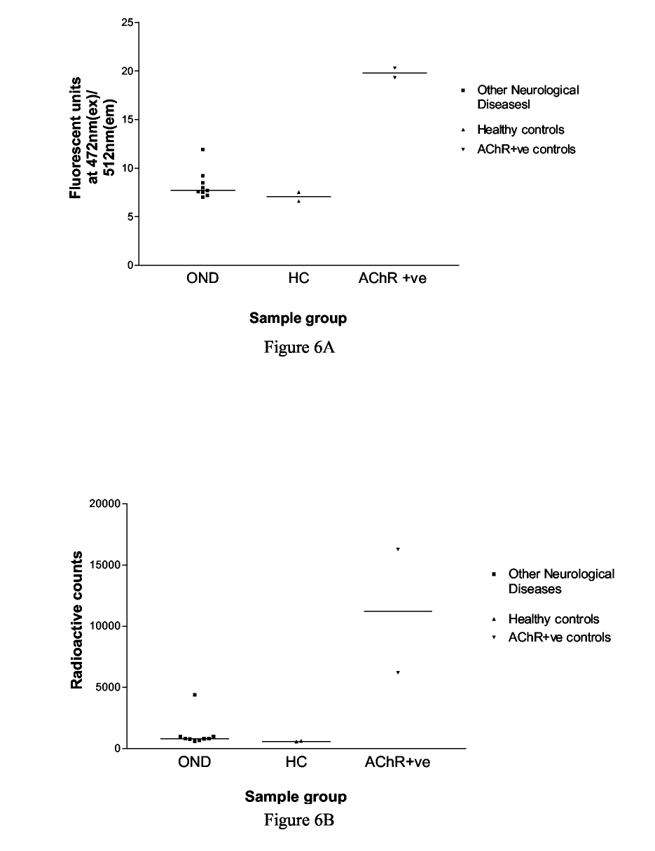

FIG. 6A shows the results of a fluorescence immunoprecipitation assay in which the protein A beads were pre-blocked by incubation with low IgG fetal calf serum (Sigma). A second set of nine “neurological disease positive” sera were assayed. One patient was determined as AChR antibody positive. In FIG. 6B, the same set of patients were assayed for AChR antibody positive sera using the standard 125I-α-bungarotoxin binding assay.

### Implications of the Results

This technique is not only applicable to the assay for myasthenia gravis but can also be used for detection of almost any defined antigen, and in particular, it could be used both for known autoimmune antigens, and as a method for screening candidate antigens in autoimmune conditions where the antigen has not yet been identified. The technique used for the labelling of the AChR in these experiments is applicable to all members of the cys-loop acetylcholine receptor ion channel gene superfamily, such as neuronal nicotinic AChRs, GABAA receptors, glycine receptors and 5HT₃ receptor. Thus the technique may be used to identify and assay for novel serum antibodies in novel patients groups. It is also applicable to any other antigen that can be fluorescent-tagged without affecting antigen binding such as labelling of muscle specific kinase (MuSK) or voltage-gated calcium, sodium or potassium ion channels. In many molecules a suitable specific radio-labelled ligand is not available. The use of the fluorescence-tagged marker, or of a tag that can fluorescence-labelled in a subsequent methodological step should provide numerous assay methods for the detection of novel antigens.

A fluorescence plate reader specifically designed for the method, apart from enhancing sensitivity, can be designed to detect emissions at a plurality of different wavelengths enabling many different antigens to be assayed in the same sample tube/experimental procedure. For example a “same tube” assay that detects and differentiates autoantibodies to AChR and MuSK would provide a single test for autoimmune myasthenia gravis.

### Example 2

### An Assay for Serum Antibodies to Muscle-Specific Kinase (MuSK) Using a EGFP-Tagged MuSK

Serum antibodies to muscle-specific kinase (MuSK) underlie a form of myasthenia gravis in which antibodies to the AChR are absent. Diagnostic tests for anti-MuSK myasthenia gravis at present are performed by radio-labelling purified MuSK with ¹²⁵Iodine, and using this radiolabelled antigen in standard immunoprecipitation assays. In a method according to the present invention, EGFP-tagged MuSK is synthesised and fluorescence-tagged immunoprecipitation is used as a method for detection of anti-MuSK antibodies in serum samples.

### Method

HEK 293 cells were transiently transfected with the plasmid pSecTagA ¹²³⁴MuSK-GFP, which is derived from the plasmid pSecTagC (Invitrogen), and which encodes the extracellular region of human MuSK (residues 22-473), a polyhistidine tag and EGFP. The secreted protein was harvested from cell growth medium (Cambrex UltraCHO) after 2 and 5 days. The protein was purified on a nickel column (Invitrogen Probond Resin) according to the manufacturers instructions, at 4° C. The eluted protein was dialysed overnight at 4° C. against PBS and subsequently concentrated using a Centriprep column (YM-50, Amicon). The purified 85 kD MuSK-EGFP protein, which contains the majority of the mature extracellular domain of the protein, was subsequently shown to be highly purified by western blot with anti-human MuSK, anti-polyhistidine tag, and anti-GFP antibodies.

To assay patient serum for the presence of anti-MuSK antibodies, 10 μl of serum was added to 10 μg of purified MuSK-EGFP and incubated overnight at 4° C. with gentle agitation. Protein A sepharose beads (FastFlow 4B, Sigma) were preincubated in 5% low IgG FCS (Sigma) for 30 mins at room temperature, to minimise non-specific binding. 75 μl of pre-treated beads were subsequently added to each assay tube and the antibody/protein complexes were left to bind to the beads for 2 hours at room temperature with gentle rotation. The protein A beads were then washed 4 times in PBS and the amounts of MUSK-GFP/antibody bound by the beads was measured by placing the beads into a conical bottom black PCR plate (Thermo-Fast 96 well black PCR plate, ABgene) and the fluorescence measured at 472 nm (excitation)/512 nm (emission) in a fluorescent plate reader (Spectra Max Gemini XS, Molecular Devices).

### Results

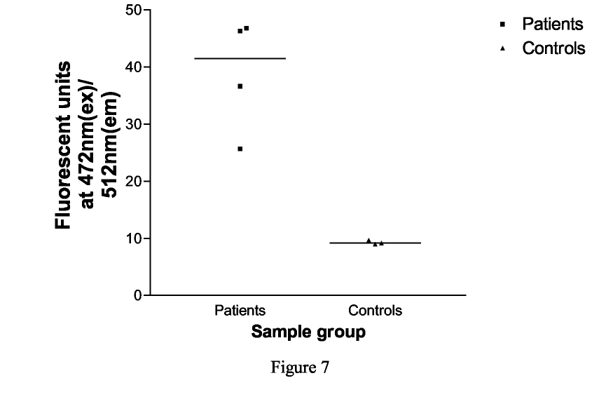

FIG. 7 shows detection of anti-MuSK antibodies in human sera. Sera (10 μl) from affected MuSK+ve patients or control individuals were incubated with purified MuSK-GFP, immunoprecipitated, and fluorescence units determined. As shown, the known patient samples all showed increased fluorescence compared to control samples with a signal at least three times the background meeting. The assay was able to clearly differentiate between control sera and samples with anti-MuSK antibodies.

### Example 3

### An Assay for Serum Antibodies to Aquaporin-4 (AQP4) Using a EGFP-Tagged AQP4

Although there are criteria laid down as a guide to differentiate between Multiple Sclerosis (MS) and Neuromyelitis Optica (NMO), they present with similar symptoms. An early difference in the pathogenesis of the two diseases seems to be the presence of detectable levels of autoantibodies to the water channel aquaporin-4 (AQP4) in about 65% of NMO patients. These autoantibodies have not been detected in MS patients. Because of the severity of NMO (Devic's disease) and the different treatment needed for the two diseases, an assay to differentiate them at an early stage would be of immense value. The principle behind this assay, which used immunohistochemical techniques, was published by Lennon et al. J Exp Med (2005) 202 (4): 473-7 and Weinshenker et al. Dis Markers. (2006) 22 (4):197-206.

Aquaporin 4 is a member of the aquaporin family of membrane water channels. It is abundantly expressed in the optic nerve and spinal chord, but found throughout the brain, predominantly located in astrocytic foot processes that abut blood vessels. Two isoforms exist, M1 (323 amino acids; where M stands for the initiation methionine) and M23 (301 amino acids) that differ by 22 N-terminal amino acids, which are found only in the M1 isoform. M23, like the other aquaporin family members, is composed of 4 exons, while M1 has an extra exon, designated exon 0, which codes for the first 11 amino acids. They form multimers of tetramers with a water channel formed by each monomer.

### Methods

### Construction of the Plasmids

Full length AQP4 cDNA was obtained from gene service (clone IMGCLO4717755). The M1 isomer was amplified from this using the following primers:

5′-gTcAcTcgAgATggTggcTTTcAAAggggTcTg
and
5′-gcATcccgggTcATAcTgAAgAcAATAccTcTccAg,

sub-cloned into pGEM-Teasy, cut out with XhoI and XmaI and cloned into the mammalian expression vector pEGFP-C3 (BD Biosciences). A similar protocol was followed for M23, but the forward primer used was

5′-gTcAcTcgAgATgAgTgAcAgAcccAcAgcAAg.

### Expression of EGFP-AQP4-M1/M23

HEK-293 cells were transiently co-transfected overnight with 1.5 μg of each of the AQP4 isomers using PEI. After 48 hr the channels were extracted in buffer containing 10 mM Tris, 100 mM NaCl, 1 mM EDTA, 1% Triton-X-100, pH 7.5, protease inhibitor cocktail (Sigma) for 1 hr @ 4° C., clarified at 15,000 rpm for 4 min and the AQP4 containing supernatant was stored at 4° C.

### Immunoprecipitation

250 μl supernatant was incubated with 25 μl serum overnight at 4° C. 50 μl Protein-A (or Protein-G) Sepharose beads (Sigma), blocked for 1 hr at 4° C. with ultra low Ig FCS, washed, added to the mixture and rotated gently for 2 hr at RT. The beads were washed thrice with 1 ml extract buffer, transferred to a conical-bottom black PCR plate (ABgene) and the fluorescence at 512 nm was read on a SpectraMAX GeminiXS plate reader (Molecular Devices) after excitation at 472 nm.

### Results

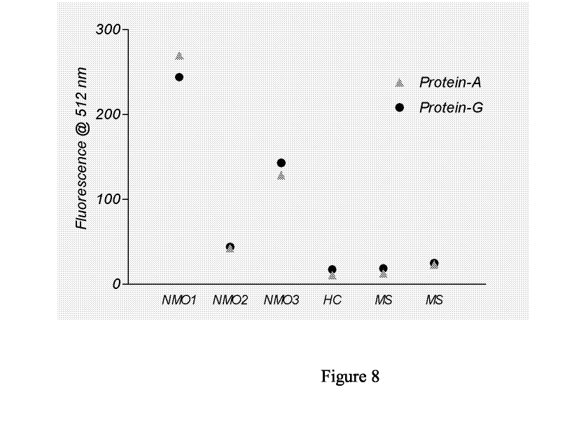

FIG. 8 shows a comparison of Protein-A (▴)v Protein-G () Sepharose beads in capturing the antibodies to aquaporin 4 in the sera from a variety of NMO and control samples. Assays performed using 25 μl of serum from 3 NMO, 2 MS and 1 healthy control individual are shown. The fluorescence is greater in each of the NMO samples than in each of the control samples (MS or healthy individual). Similar results were obtained using Protein-A or Protein-G Sepharose beads.

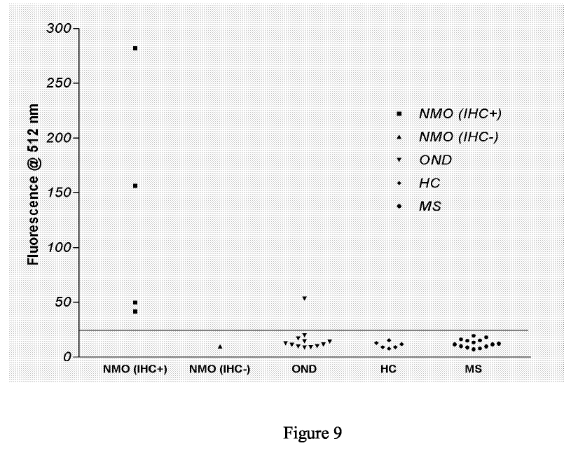

FIG. 9 shows an assay for antibodies to AQP4 in the sera from patients with NMO, other neurological diseases, healthy controls and MS (IHC+: sera tested positive on immunohistochemistry, IHC−: sera tested negative on immunohistochemistry). Each of the NMO IHC+ samples had a fluorescence reading at 512 nm above about 25. In contrast all of the remaining samples had a fluorescence reading at 512 nm below about 25, except for on sample from a patient with an “other neurological disease”.

1. A method for detecting antibodies against a target antigen in a sample which comprises contacting the sample with labelled target antigen, subjecting the sample to immunoprecipitation to precipitate antibodies in the sample and detecting the presence of antibodies against the target antigen in the sample by means of the presence of labelled target antigen in the immunoprecipitate, wherein the labelled target antigen is a fusion protein comprising the target antigen and a fluorescent protein label and the presence of labelled target antigen in the immunoprecipitate is detected by means of the fluorescence of the fluorescent protein label.

2. A method according to claim 1 wherein the amount of antibody is determined by quantitating the fluorescence in the immunoprecipitate.

3. A method according to claim 1 wherein the sample is a serum sample.

4. A method according to claim 1 wherein the target antigen is an autoantigen.

5. A method according to claim 1 wherein the fluorescent protein label is derived from GFP.

6. A method according to claim 5 wherein the fluorescent protein label is EGFP, ECFP or EYFP.

7. A method according to claim 1 wherein the target antigen is:
a protein which is a member of the cys-loop acetyl choline receptor ion channel gene superfamily;
a protein which is a member of the voltage-gated calcium, sodium or potassium ion channel gene superfamily;
a protein which is a member of the glutamate receptor gene family; or
a receptor tyrosine kinase.

8. A method according to claim 7 wherein the target antigen is:
a neuronal nicotinic AChR, GABAA receptor, glycine receptor or 5HT₃ receptor;
Kv1.1-Kv1.7 (or KCNA1-KCNA7);
GluR1-GluR4, a kainate receptor or NMDA receptor; or
a muscle specific kinase or growth factor receptor;
or a protein which is a member of the aquaporin gene family

9. A method for identifying autoantigens implicated in an autoimmune disorder which comprises screening serum samples from patients with a clinical phenotype indicative or suggestive of an autoimmune disorder and suitable controls with a labelled putative autoantigen, subjecting the samples to immunoprecipitation to precipitate antibodies therein and identifying actual autoantigens by the presence of the labelled putative autoantigen in the immunoprecipitate, wherein the labelled putative autoantigen is a fusion protein comprising the putative autoantigen and a fluorescent protein label and the presence of labelled putative autoantigen in the immunoprecipitate is detected by means of the fluorescence of the fluorescent protein label.

10. A method according to claim 9 wherein the amount of autoantibody is determined by quantitating the fluorescence in the immunoprecipitate.

11. A method according to claim 9 wherein the fluorescent protein label is derived from GFP.

12. A method according to claim 11 wherein the fluorescent protein label is EGFP, ECFP or EYFP.

13. A reagent suitable for use in screening samples for antibodies against a target antigen comprising a fusion protein comprising the target antigen and a fluorescent protein label, wherein the target antigen is:
a protein which is a member of the cys-loop acetyl choline receptor ion channel gene superfamily;
a protein which is a member of the voltage-gated calcium, sodium or potassium ion channel gene superfamily;
a protein which is a member of the glutamate receptor gene family; or
a receptor tyrosine kinase.

14. A reagent according to claim 13 wherein the target antigen is:
a neuronal nicotinic AChR, GABAA receptor, glycine receptor or receptor;
Kv1.1-Kv1.7 (or KCNA1-KCNA7);
GluR1-GluR4, a kainate receptor or NMDA receptor; or
a muscle specific kinase or growth factor receptor.

15. A reagent according to claim 13 wherein the fluorescent protein label is derived from GFP.

16. A reagent according to claim 15 wherein the fluorescent protein label is EGFP, ECFP or EYFP.
